(19) United States
(12) Patent Application Publication
(10) Pub. No.: US 2016/0361010 A1
(43) Pub. Date: Dec. 15, 2016

(54) Real time parformance data and biofeedback data visually displayed on the interior of swim goggles

(71) Applicants: Lambert Gregory Graham, Sammamish, WA (US); Xavier W. Graham, Sammamish, WA (US); Ivan W. Graham, Sammamish, WA (US)

(72) Inventors: Lambert Gregory Graham, Sammamish, WA (US); Xavier W. Graham, Sammamish, WA (US); Ivan W. Graham, Sammamish, WA (US)

(21) Appl. No.: 14/545,734

(22) Filed: Jun. 12, 2015

(51) Int. Cl. A61B 5/00; A63B 33/00; G02B 27/01; A61B 5/024

(52) U.S. Cl. CPC A61B 5/486; A61B 5/4866; A61B 5/02438; A63B 33/002; G02B 27/0176; A63B 2033/004; G02B 2027/0178

(57) ABSTRACT

Real time data is the quickest method see if changes made in technique are being translated into better swim performance. Swimmers can see in real time the positive or negative benefits of changing their tempo, breathing, speed, or stroke technique. Real time data projected to the interior of the goggle lenses allows the swimmer instant feedback on their performance so that a more proficient overall technique can be attained by bold or subtle changes the swimmer initiates while in the pool. The design integrated into the goggle will be less obtrusive to the swimmer.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

Currently the most common performance and or biofeedback information for swimmers is done after the fact. In the case of bio feedback, pulse rate, temperature, oxygen levels, are taken after the swim is finished. The performance data generated is similarly accumulated outside of the swimmers real time view.

### BRIEF SUMMARY OF THE INVENTION

An integrated design with goggles allows data generated to be viewed by the wearer in real time. To be used by the wearer to adjust and improve swimming ability. This integrated arrangement allowing for real time data stream to be viewed by the wearer allows for precise real time changes in breathing, stroke, body rotation, muscle group use and tempo. Previously any changes by swimmers in these areas where tested over long time periods, i.e. days, weeks and months.

### BRIEF DESCRIPTION OF THE SEVERAL VIEWS OF THE DRAWING

FIG. 1 Left view of the goggles showing the placement of the integrated waterproofed processor/transmitter/battery integrated pack, the waterproofed LED pack, and the waterproofed biofeedback pack placement and focus lense.

FIG. 2 Right view of the goggles showing the placement of the integrated waterproofed processor/transmitter/battery integrated pack, the waterproofed LED, and the waterproofed biofeedback placement and focus lense.

FIG. 3 Composition of the integrated waterproofed processor pack.

FIG. 4 Composition of the integrated waterproofed biofeedback pack.

FIG. 5 Composition of the integrated LED pack.

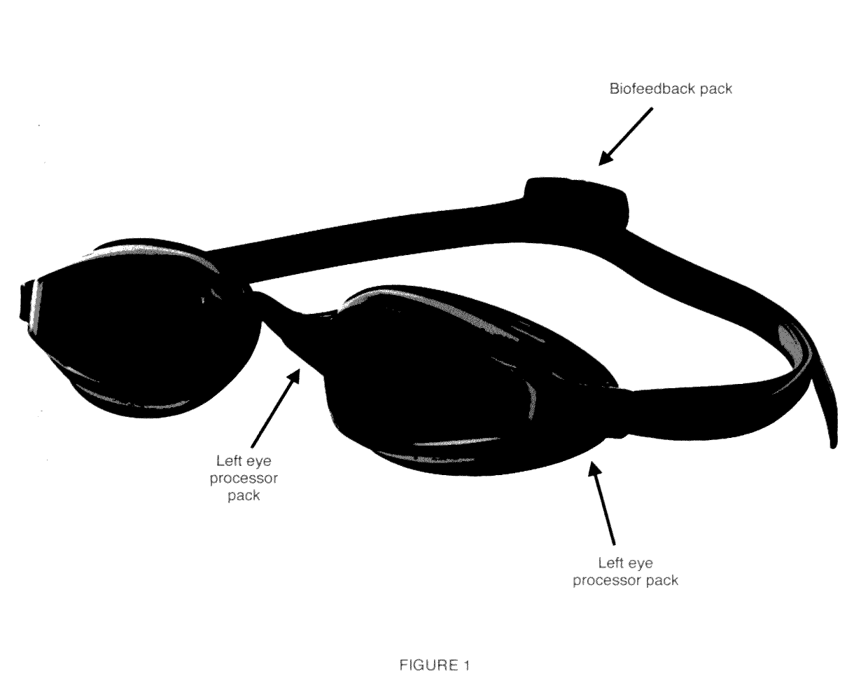

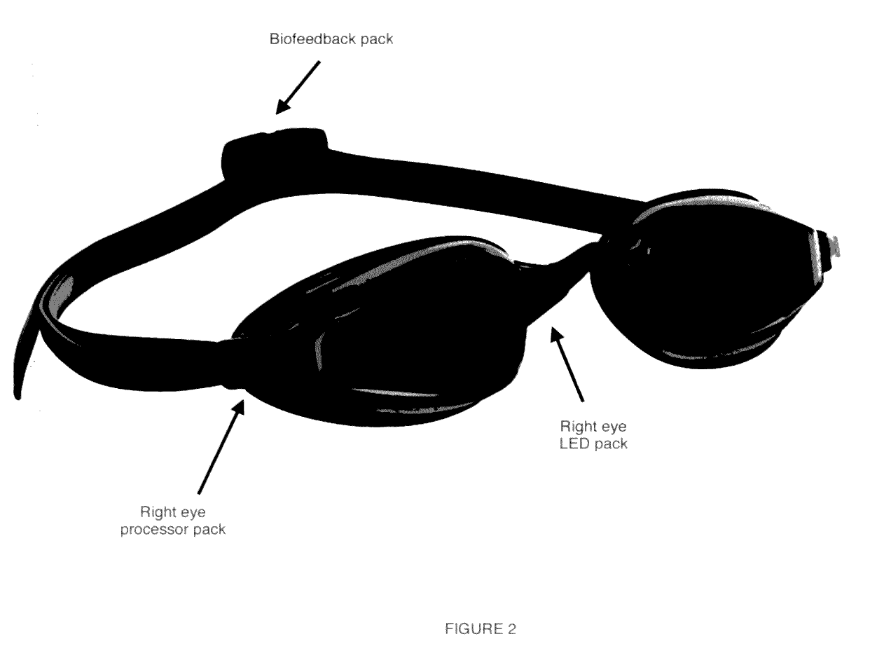

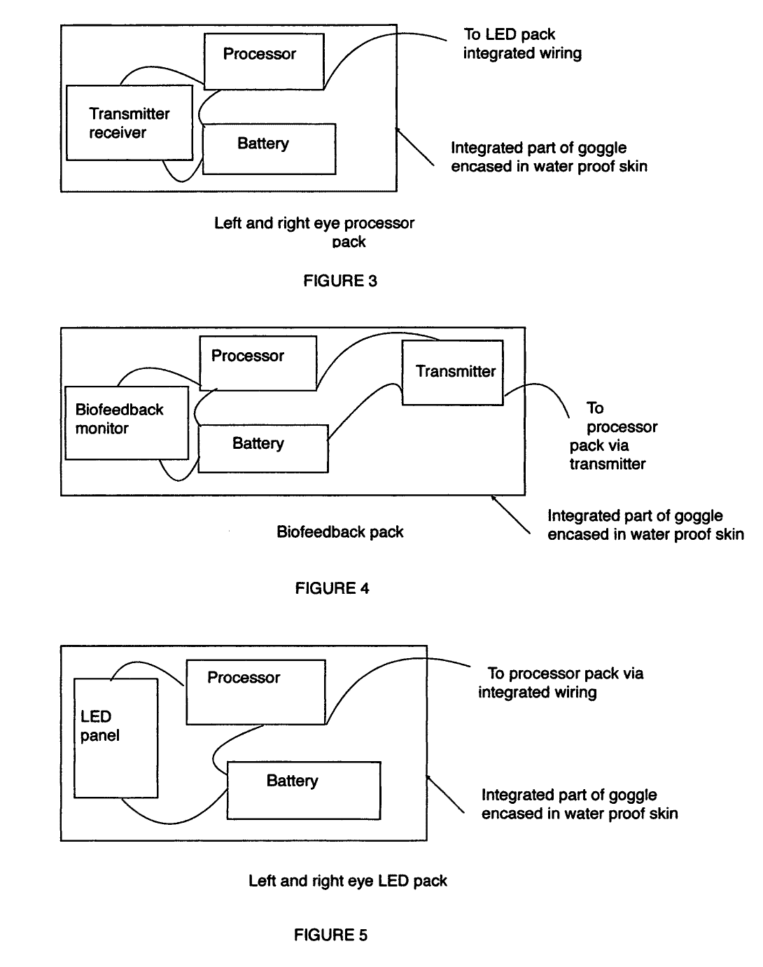

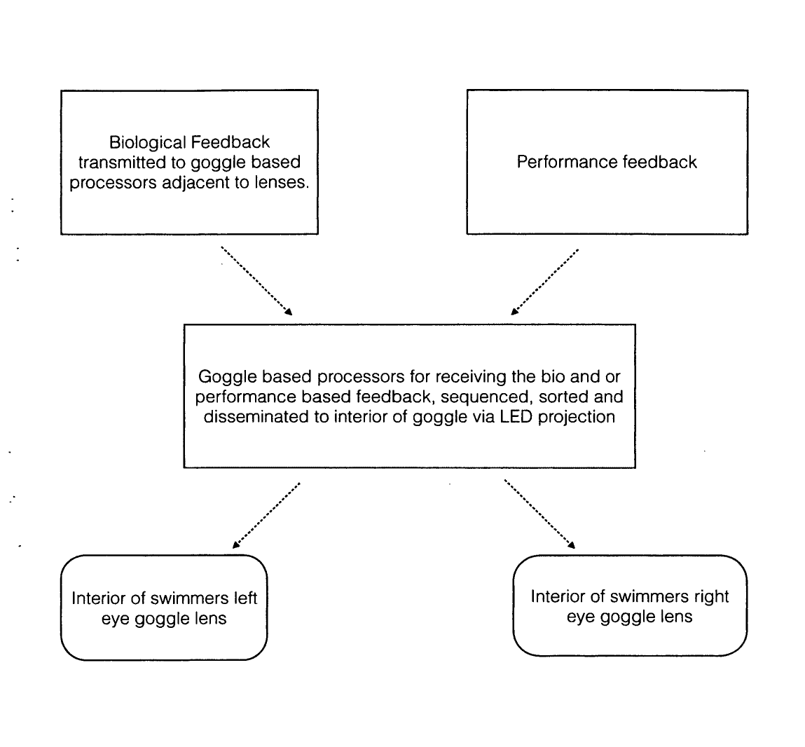

### DETAILED DESCRIPTION OF THE INVENTION

This integrated goggle and feedback processor unit design will provide real time data to swimmers as to their individual energy consumption, oxygen level, adrenalin level, pulse rate, and real time performance data regarding swimmers start time, strokes, speed, breathing pace, muscle group activity, and end of pool turn times. Real time data is projected onto the interior of the swimmers goggles via an integrated water proofed design. The data feed can be manipulated via a switch for individual data or a rotating data stream. The water proofed micro and nano hardware for data transmittal, gathering and projecting is contained within the redesigned swim goggles. The processors, data stream switch, wifi receptors, nano batteries, and nano LEDs are located at the outside edge of both lenses encased within the goggle design in a waterproof casing. At the rear of the goggle strap the biofeedback monitor, processor, nano battery and wifi transmitter is located in a goggle integrated waterproofed case. The nano LEDs integrated into the nose piece and located at the inside edge of both lenses project the processed data to the interior inside edge of both lenses. This information is displayed as a color scaled code, numerical or alphabetized information. This portion of the lenses are also adjusted in manufacture to allow close vision acuity.

1. Real time bio feedback data for swimmers.

2. Real time data for swimmers to adjust and improve their strokes rhythm.

3. Real time data for swimmers to see laps completed.
